(19) United States
(12) Patent Application Publication
(10) Pub. No.: US 2015/0283371 A1
(43) Pub. Date: Oct. 8, 2015

(54) SUBCUTANEOUS CONTROLLED DELIVERY SYSTEM FOR THE TOPICAL ADMINISTRATION OF DRUGS, BIOLOGICAL AGENTS OR THERAPEUTIC AGENTS TO TARGETED AREAS WITHIN THE BODY

(71) Applicant: Jaishankar Raman, Chicago, IL (US)

(72) Inventor: Jaishankar Raman, Chicago, IL (US)

(73) Assignee: Topicare, Inc., Oak Brook, IL (US)

(21) Appl. No.: 14/245,279

(22) Filed: Apr. 4, 2014

(51) Int. Cl. A61M 39/02

(52) U.S. Cl. CPC A61M 39/0208; A61M 2205/04; A61M 2205/3317; A61M 2205/3584

(57) ABSTRACT

The present invention is directed to a subcutaneous controlled delivery device that provides for a refillable reservoir port, a release mechanism and tubing that operably connects the reservoir port to the release mechanism. Such a device is capable of being implanted under the dermis of the skin and allows for the controlled release of drugs, biological agents or therapeutic agents to a target site of interest in the body for treatment.

### CROSS-REFERENCE TO RELATED APPLICATIONS

This application is a continuation of U.S. patent application Ser. No. 13/716,826 filed Dec. 17, 2012 which claims priority to U.S. Provisional Application Ser. No. 61/576,638, filed Dec. 16, 2011, the entire disclosure of each which is incorporated herein by this reference.

### BACKGROUND OF THE INVENTION

1. Field of the Invention

The present invention is generally related to medical implant devices, and more specifically to a subcutaneous medical implant device having a refillable reservoir that allows for accurate time release and targeted delivery of drugs, biological agents or therapeutic agents to a target site which is suitable for implantation under the skin and delivers treatments to various parts of the body.

2. Description of the Prior Art

Implantable infusion pumps that dispense medication from one or multiple compartments are known in the art. With increasing interest in the development of new drugs targeted specifically for tissues related to specific health issues, such implantable devices with the option of refilling are in high demand. The ability to control and directly deliver drugs to targeted areas of the body, without affecting the rest of the body results in improved benefits and reduced side effects for the patient. Elastomers have been used for some time to slowly release pharmacological agents into the body, for purposes of pain management with local anesthetics after surgery.

Other examples such as drug eluting stents to prevent endothelial overgrowth as vascular injury treatment deliver agents in the region of the stent. Currently available implantable drug delivery devices consist of a pump and valve systems actuated to release therapeutic agents. The use of remote control devices to control the release of drugs from outside the body is also available. However, the drawback of such existing devices is that they are not able to precisely deliver and target the specific area of the body requiring treatment in a controlled manner. They are not completely self-contained and are without the ability to be re-filled.

Since most drug and therapeutic agents are administered orally, intravascularly or occasionally directly into the organ or target in question, there is a need for a device that will allow for subcutaneous administration of drugs, biological agents (i.e., live cells such as stem cells) or therapeutic agents which are transported from a subcutaneous reservoir port to an implanted release mechanism located at or near the target area in the body. Such a device is particularly important for avoiding drug abuse by providing site directed, controlled release, drug therapy treatments.

### BRIEF DESCRIPTION OF THE DRAWINGS

FIG. 1 is a schematic of the subcutaneous delivery device of the present invention having a subcutaneous refillable reservoir port, a dispersive bladder, and a tubing to connect the port to the bladder.

FIG. 2. is a schematic of the subcutaneous delivery device of the present invention for use in connection with pain relief therapy for the lower back.

FIG. 3 is a schematic of the subcutaneous delivery device of the present invention for use in connection with cardiac therapy such as arrhythmia management and stem cell growth factor infusion.

FIG. 4 is a schematic of the subcutaneous delivery device of the present invention for use in connection with therapy for resistant hypertension.

### DETAILED DESCRIPTION

The present invention is capable of embodiments in many different forms. Preferred embodiments of the invention are disclosed with the understanding that the present disclosure is to be considered as exemplifications of the principles of the invention and are not intended to limit the broad aspects of the invention to the embodiments illustrated.

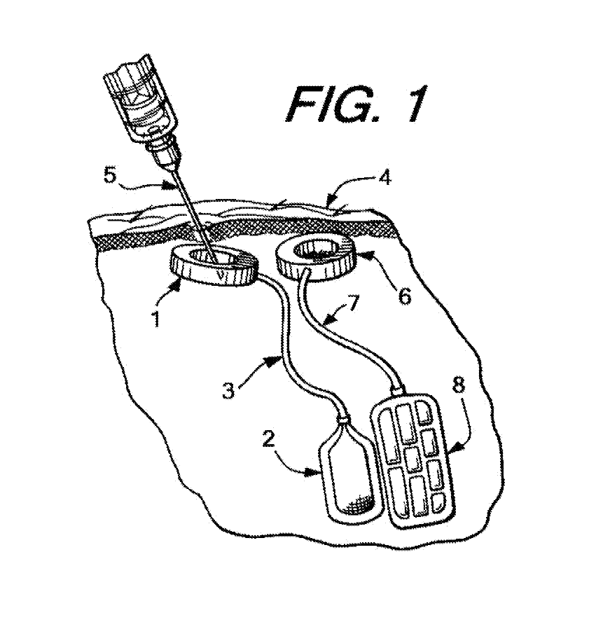

FIG. 1 discloses a subcutaneous delivery device of the present invention that is capable for use with a number of different organs and tissues throughout the body. The delivery device generally comprises a subcutaneous refillable reservoir port, tubing, and a release mechanism. In one embodiment of the present invention, the subcutaneous delivery device comprises a reservoir port 1 that is operably connected to a release mechanism 2 by a fluid transfer system 3 such as tubing. The reservoir port, fluid transfer system and release mechanism are implanted under the skin and preferably beneath the dermis layer of the skin of a subject. In another embodiment, the port is subcutaneous but the tubing traverses other tissue to allow the delivery component to sit in a joint, over an organ, adjacent to resected tumor margins, etc.

As shown in FIG. 1, the reservoir port 1 is positioned beneath the surface of the skin 4 of a patient, and is therefore accessible by a hypodermic needle 5. The reservoir port 1 is capable of holding a predetermined amount of drugs, biological agents or therapeutic agents and can be refilled as desired. In one embodiment of the present invention, multiple reservoir ports or release mechanisms are contemplated for use in administering more than one therapeutic agent for treatment. As shown in FIG. 1, an alternate embodiment of the invention having a second reservoir port 6, fluid transfer system 7 and release mechanism 8 is provided. It is contemplated that the release mechanism 2 and the release mechanism 8 may be different so as to accommodate differing treatments.

The release mechanisms 1 and 8 depicted in FIG. 1 may be in the form of dispersive bladders, each of which consists of a flexible mesh or network of tubing that functions to allow for controlled delivery of drugs, biological agents or therapeutic agents over a wide area such as an organ. The pore size of the bladders, tubing or meshwork can be adjusted to be specific for the kind of drug to be delivered. Thus, it is contemplated that the release mechanism may be integrated with the fluid transfer system, for example, as perforations in a fluid transfer tube. Alternately, the release mechanism may include macroscopic, microscopic, or nanoscale release functions, depending on the type of reagent, drug, or cells to be delivered to the site. For example, the release mechanism could comprise, but is not limited to, a slow release bladder, mesh like tubing constructs or other tubing having the same of differing perforations along it. The overall structure of the each particular release mechanism will vary in size and shape depending on the intended application (i.e., depending on whether it is to cover a large portion of an organ or if it is being used to treat a small tissue area).

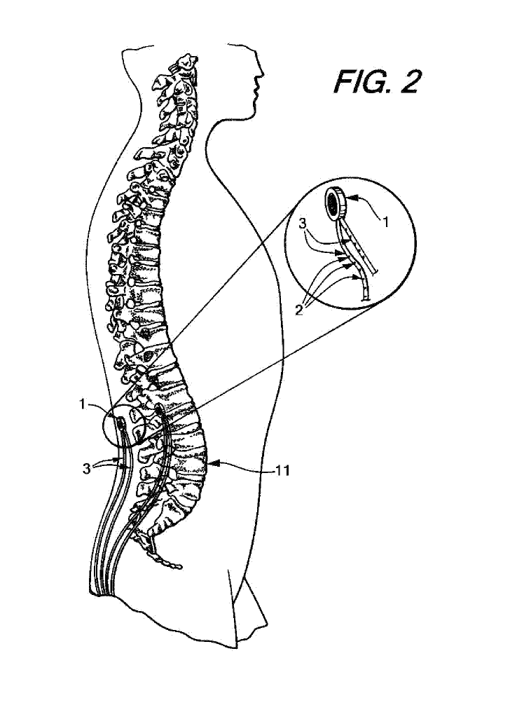

As shown in FIGS. 2-4, various therapy applications are contemplated by the present invention, including but not limited to, pain relief treatments, cardiac arrhythmia management, stem cell growth factor infusion and treatments for resisting hypertension. In one embodiment, the release mechanism is located in close proximity to the site of treatment. As shown in FIG. 2, the delivery device is positioned under the skin along the spine of an individual. The reservoir port 1, shown also in an exploded view, is positioned in the upper lumbar area, for example, and the fluid transfer system 2a and 2b extend along the lower spine 11. Integrated with the fluid transfer system is the release mechanism, shown as microscopic pores which agents may be dispersed through.

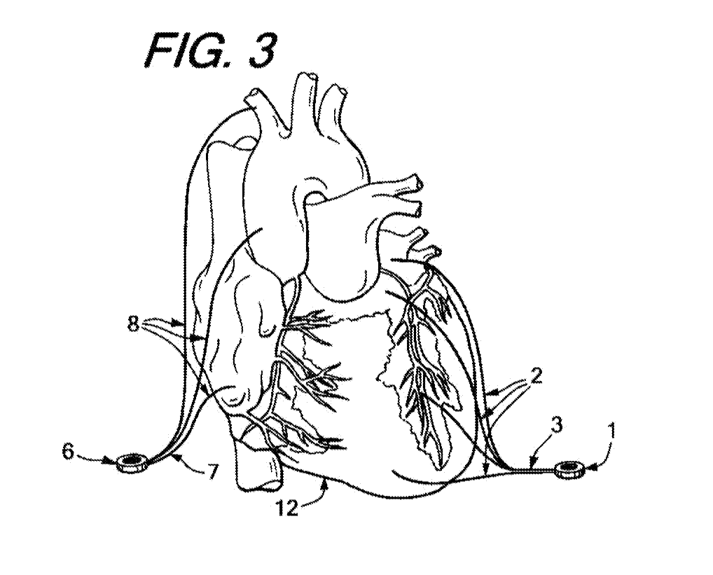

As discussed above, the delivery device is implanted beneath the skin, and may be situated in close proximity to an organ, such as a heart 12 shown in FIG. 3, requiring treatment. It is further contemplated that release mechanism may overlie a scar in the heart which has stem cells injected into it for treatment. Consequently, the pore size, extent and speed of delivery, area of delivery and other parameters for variable delivery can be precisely controlled. Remote access diagnostics may be built into the delivery device using site-specific sensors for feedback on function and necessary adjustments. This would incorporate diagnostics and therapeutics in the same system—also known as Theragnostics. Feedback loops could be used to control dosage delivery based on local diagnostic parameters.

A fluid transfer system, such as tubing, is provided to operably connect the reservoir port to the release mechanism. The tubing is used to transport the agents from the reservoir port directly to the release mechanism thereby allowing for controlled delivery of a therapeutic agent to the specific target site for treatment. In one embodiment, the tubing may comprise microscopic pores along the length of the tubing for easy delivery of therapeutic agents. In some circumstances the site of treatment may be over a wide area, such as an organ. For certain applications all three components, namely, the reservoir port, the tubing and the release mechanism, can be combined into one compact implant device which is capable of fitting into a small integrated space.

As discussed above, one aspect of the present invention is the ability to use an external control to release and stop, and therefore properly dose, the drugs or agents at specific times by an external device located either at the physician's office or patient's home. For such applications the reservoir port may contain a transmitter/receiver that will control a valve based on a charge or magnetic driver that allows or disallows the flow of agents through tubing to the implanted release mechanism. Also, at the site of the reservoir port, an additional device could be included that allows sensing through cantilever technology, electrochemical or magnetic sensor to measure the efficacy of treatment and provide feedback to an outside control unit.

While the present invention has applications for various purposes, several of those applications are discussed below. In one embodiment a subcutaneous delivery device can be implanted in the vitreous cavity of the eye, or at the surface of the eye/retina, to slowly release drugs such as, but not limited to, triamcinolone to decrease macular edema due to diabetic maculopathy. Conventional injections need to be repeated due to the transient effect of triamcinolone resulting in possible complications such as cataract, steroid-induced glaucoma and endophtalmitis. But the present invention allows for a more precise dosing regimen over time using a slow release, controlled implant treatment. Alternatively, for treatment of diabetic retinopathy, oligomeric proanthocyanidins can be released by the subcutaneous delivery device to improve microcirculation, retinal edema and visual acuity. New drugs such as anti-angiogenics or anti-VEGF (anti-Vascular Endothelial Growth Factor) agents can be released using the implanted reservoir (as opposed to traditional multiple injections) into the vitreous humor to regress abnormal blood vessels and improve vision in wet macular degeneration.

In a compact form, the subcutaneous delivery device of the present invention can provide slow and controlled release of antibiotics in the middle ear region to treat chronic otitis media.

Alternatively, the device may be designed to allow for a renewed supply of stem cells in stem cell therapy. For this purpose the release mechanism could consist of a polymer matrix that supplies stem cells from the subcutaneous reservoir and delivers such stem cells at the interface of the polymer matrix and the area of treatment. Multiple release mechanisms that include both stem cells and a separate release channels such as growth factors for treatment of such stem cells could be included.

Another embodiment of the present invention includes a tube shaped flexible delivery mesh that can be surgically positioned around the colon for delivery of therapeutic agents in patients with inflammatory bowel diseases such as Crohn's and Ulcerative Colitis. The mesh like structure may consist of tubing with slow and controlled release pores, or linked sets of small sheet structures with controlled release mechanisms for releasing anti-inflammatory and/or immunosuppressing agents. Where the subcutaneous reservoir is connected to sheet like release structures it could also be implanted at or near joints for local treatment using anti-inflammatory drugs for conditions such as inflammatory arthritis, degenerative joint disease, gout, or other ailments effecting joints at various locations in the body.

It is further contemplated that the delivery device is adapted for implantation near or at different surfaces of the heart as shown in FIG. 3. For instance, pharmacological treatment of postoperative atrial fibrillation can be applied locally to convert to a regular synus rhythm, and antibiotic therapy can be administered locally at the site of implanted cardiac devices such as ventricular assist devices. Some of these devices can be re-absorbable over time (3, 6, 9 months—similar to suture material like polygalactin). Alternatively, the disclosed subcutaneous delivery system can deliver live cells that can be used to supply human CD34+ stem cells that induce neovascularization in ischemic myocardium enhancing perfusion and function.

The delivery device can be implanted at or near the genitourinary system components such as the bladder or prostate. Doing so will allow for local delivery to remedy prostatic or bladder malignancies. Depending on the location, a polymer mesh or bladder will be used for slow and controlled release of pharmacological agents.

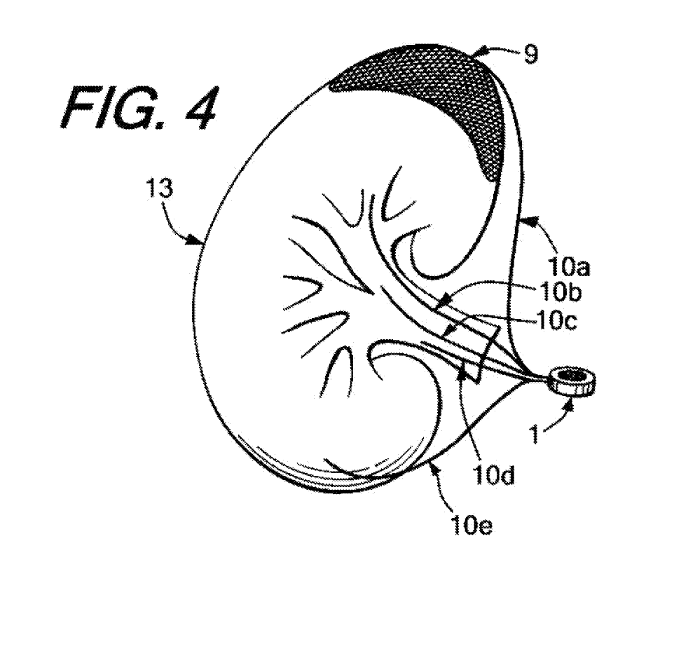

For use in organ transplant applications, the release mechanism is simultaneously implanted during organ transplantation to allow for immunosuppressive and antibiotic therapy directly at the site of transplantation. This could involve the mesh like release mechanism to be positioned around the transplanted organ, or a flexible bladder implanted into or adjacent to such organ. For example, in the case of the treatment being applied to a kidney 13, as shown in FIG. 4, the release mechanism in the form of a mesh 9 may cover the portion of the organ where agents must be provided. Furthermore, flexible tube shaped mesh structures 10a, 10b, 10c, 10d, 10e, can be applied around sites of vascular anastomosis for topical therapy, which is one of the key aspects in organ transplantation.

As discussed above, the delivery device may be customizable and could therefore be constructed to for re-absorption over time, such as 3-6 months for peri-operative issues with cardiac surgery where peri-vascular anti-inflammatory agents are administered to reduce the risk of anastomotic narrowing.

Another version will have a slow release polymer attached to plates and screws that have bone growth stimulating factors such as BMP (bone morphogenic protein), to act in bone healing in patients undergoing fixation of bones in fractures. This would be very pertinent to patients with osteoporosis and osteopenia, where local delivery of alendronates or calcium handling proteins may aid in strengthening those bones. The further corollary would be to implant local bone strengthening medications by providing a delivery device overlying the bone that needs build up—as in the neck of the femur or the body of the vertebrae, after a fracture.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the following claims.

What is claimed is:

1. A subcutaneous delivery device comprising:
a reservoir port;
a release mechanism; and
tubing that operably connects the reservoir port to the release mechanism to administer drugs, biological agents or therapeutic agents to a target treatment site in a controlled manner.

2. The device of claim 1, wherein the reservoir port is implanted in a small area near the dermis.

3. The device of claim 1, wherein the reservoir port has a membrane through which the agents are injected.

4. The device of claim 1, wherein the tubing transports the agent from the reservoir port to the site of treatment.

5. The device of claim 1, wherein the release mechanism comprises macro-, micro, or nanopores for local treatment.

6. The device of claim 1, wherein the release mechanism comprises polymeric membranes or silicon based porous surfaces.

7. The device of claim 6, wherein the polymeric membranes are interfaced with 3-dimensional mechanical tissue engineering constructs for release and sustenance of stem cells at the target site for treatment.

8. The device of claim 1, wherein the release mechanism is customizable depending on the desired treatment.

9. The device of claim 1, wherein the reservoir port includes a wireless transmitter/receiver to relay data between the device and a control unit located outside the body.

10. The device of claim 9, wherein the control unit triggers operation of the release mechanism and/or reservoir port.

12. The device of claim 1, wherein the release mechanism at the target site includes a sensor for relying information from the target site to the control unit.
